(19) United States
(12) Patent Application Publication
(10) Pub. No.: US 2012/0101418 A1
(43) Pub. Date: Apr. 26, 2012

(54) SEMI-RIGID COMPRESSION SPLINT FOR APPLICATION OF THREE-DIMENSIONAL FORCE

(75) Inventors: Seth M. Manoach, New York, NY (US); Elton Strauss, East Hills, NY (US)

(73) Assignee: The Research Foundation of State University of New, Albany, NY (US)

(21) Appl. No.: 13/058,955

(22) PCT Filed: Aug. 13, 2009
(86) PCT No.: PCT/US09/53670

§ 371 Date: Jul. 1, 2011

Related U.S. Application Data
(60) Provisional application No. 61/088,641, filed on Aug. 13, 2008.

(51) Int. Cl. A61F 5/00

(52) U.S. Cl. 602/13

(57) ABSTRACT

A compression splint is provided for reduction, compression and stabilization of spinal, pelvic, and long bone fractures. The compression splint includes a plurality of plates selectively disposed at a plurality of positions of a fracture site; a plurality of inflatable cushions, each cushion positioned between respective plates of the plurality of plates and the fracture site; and a support means for supporting the plurality of plates and plurality of cushions compressively against the fracture site in three dimensions, Additionally, the plates of the compression splint can be adjusted, removed or additional plates added to further customize the compressive force applied to the fracture site.

### FIELD OF THE INVENTION

The present invention relates generally to compression splints for trauma and, more particularly, to an axial, rotation, semi-rigid compression splint.

### BACKGROUND OF THE DISCLOSURE

Bone fractures are stabilized with splints until proper diagnosis and treatment are available. Medical personnel align a fractured bone, which is referred to as reduction, and maintain the alignment through various treatments for healing. The following presents approaches to noninvasive reduction of fractures of the pelvis and long bones.

Compression splints of the prior art are typically fabricated from a solid piece of fabric or other material. While these splints have their advantages, in order to ensure that the providers can see and access the area being treated by the splint, such as the spine, limbs, anterior abdomen, pelvis, and ano-genital regions, the splint must be disengaged. Furthermore, pelvic compression splints of the prior art cause compressive forces to be applied to the soft anterior abdominal/pelvic wall and not to the bony pelvis itself where it is most needed

For example, the invasive external pelvic compression/stabilization devices of the prior art include the External Fixator, C-Clamp, and Pelvic Stabilizer. The term “invasive” is understood herein to mean that in order to use any of these devices, an orthopedic surgeon, or other specially trained provider is required to make multiple incisions in the skin and soft tissue dissections in order to properly attach the device.

The devices mentioned above are all designed to stabilize and compress the pelvis before definitive operative repair can be done and primarily provide medially-directed compression of the pelvis while allowing the trauma team to have access to the abdomen and extremities. As briefly mentioned above, all work on the same principle: an external compression frame is connected to the pelvis by means of transcutanous pins that are surgically placed into the iliac wings or posterior ileum. The frame is then adjusted to compress/stabilize and possibly reduce the pelvic disruption. The frame itself can then be swung inferiorly or superiorly to facilitate access to the abdomen or lower limbs.

The external compression frames have two major advantages, they are effective in compressing pelvic fractures, and they allow unrestricted access to the abdomen or lower extremities. Despite these advantages, use is severely limited by the need to have an orthopedic surgeon present that can dissect through skin and muscle and properly place a pin in bone. Obviously, the required time and talent for this procedure is not available in the pre-hospital context, and rarely, if ever, available in a community hospital that functions in the trauma system as a point for resuscitation, stabilization, and transfer.

Finally, in the situation in which an external fixator or C-Clamp is desired, the in-house orthopedic staff often consists of residents who are not as highly skilled in performing the procedures as attending orthopedic traumatologists with expertise in this area. Even with skilled staff there are other costs of placing the frames. For example, a risk exists that a pin tract infection will complicate later open reduction and internal fixation. Moreover time may be lost for other emergent procedures, such as diagnostic peritoneal lavage, computed axial tomography (CT) scan, laparotomy and pelvic angiography. Additionally, not all fractures can be treated with an external fixator or C-clamp. If the fracture is too communicated, i.e. in too many pieces, these devices cannot be effectively utilized.

The semi-rigid pelvic compression splint disclosed in U.S. Pat. No. 6,626,856 issued to Manoach (herein after, “Manoach”), an inventor of the present invention. It provides for a pelvic compression splint having two rigid plates with inflatable bladders for providing compressive lateral force on the pelvis. However, as noted in Manoach, the splint disclosed in U.S. Pat. No. '856 inhibits examination of the posterior side of the pelvis once the splint has been applied to a patient, thus care must be taken to examine the posterior side of the pelvis before use.

Each device that is used to compress and stabilize a fracture, be it pelvic, spine or long bone, has unique attributes and flaws. The principal devices in common use are abdominal binders, and a variety of external frames that use pins to attach to the fractured bone. The semi-rigid compression splint of the present invention is designed to combine non-invasiveness and adjustability, with medially directed compression vectors and limb, femoral and anterior abdomino-pelvic exposure.

In contrast to the use of non-invasive compression splints for stabilizing pelvic fractures, long-bone fractures are reduced by manipulating the fracture segments into proper alignment. The reduced fracture is surgically repaired or encased in a cast. There are no noninvasive means, other than manually applied force, to reduce a long-bone fracture.

### SUMMARY OF THE DISCLOSURE

The semi-rigid compression splint for trauma is a new device that combines the in-field simplicity of use, non-invasiveness and access to the fracture site that is lacking in the conventional fracture treatment technologies. It is an inexpensive, completely non-invasive, rapidly-applied splint that will provide reduction of long bone fractures or pelvic compression and stabilization at the sacro-iliac joint and the pubic symphysis. No specialized training is required to apply the splint, and can be applied during pre-hospital stabilization at an accident scene, for example, by emergency medical technicians (EMT) of an emergency medical services (EMS) or other first responders, such as fire and police personnel. The present invention can be applied in an emergency department (ED), operating room (OR) or intensive care unit (ICU) setting as well. Even when applied and fully engaged, the splint allows visualization of the injured limb and in the case of the fractured pelvis access to the ano-genital and femoral areas, anterior abdomen and pelvis, and lower limbs. Moreover, the semi-rigid compression splint of the present invention has been found to have no deleterious effects on cardio-pulmonary mechanics. The semi-rigid compression splint has an adjustable attachment and compression system, can be left in place during laparotomy, and is designed to minimize skin complications.

It should be noted, in the context of the present disclosure the term “compression” encompasses volume compression as applied to pelvic fractures, as well as reduction and alignment of fractures as applied to long bone fracture treatment. Thus, the term “compression” as used herein is intended to encompass the broader meaning of application of pressure to a site.

The semi-rigid compression splint of the present invention is designed to be easier and faster to place on the patient during the transport and resuscitation process. It allows the physician to see and access the fracture site, and provides the medially directed and rotationally directed compression vectors most likely to achieve stabilization, compression and partial reduction of the fracture. The compression splint of the present invention can be used in emergency departments, operating rooms, CT Scan or radiology departments, and intensive care departments.

Accordingly, a compression splint for splinting support and compression of the pelvis and other extremities, including thoracic and lumbar spine, and upper and lower limbs is provided. The compression splint includes a plurality of plates selectively disposed at a plurality of positions in proximity to a bone fracture; a plurality of cushions; and a support means for supporting the plurality of plates and plurality of cushions compressively and rotationally against the area being treated in three dimensions.

Each cushion of the plurality of cushions is positioned between respective plates of the plurality of plates and the area being treated. In the preferred embodiment of the present invention, a number of plates of the plurality of plates are removable and additional plates can be added as necessary during application of the compression splint to a patient.

### BRIEF DESCRIPTION OF THE DRAWINGS

These and other features, aspects, and advantages of the apparatus of the present invention will become better understood with regard to the following description, appended claims, and accompanying drawings where:

FIG. 1 illustrates for orthogonal views of a rigid plate assembly of an embodiment of the present invention;

FIG. 2 illustrates a top and bottom view of a strap component of an embodiment of the present invention;

FIG. 3 illustrates an assembled compression splint in accordance with an embodiment of the present invention;

FIG. 4, illustrates a perspective view of the rigid plate assembly of FIG. 1; and

FIG. 5 illustrates a method for using an embodiment of the compression splint for reducing and stabilizing an extremity fracture.

### DETAILED DESCRIPTION OF DISCLOSURE

A semi-rigid compression splint of the present invention will be discussed first in general as applied to a fracture and below with more specificity. The splint, when in an open position is slid posterior to (under) the supine patient's buttocks, when applied to a pelvic fracture, or placed under the patient's head, neck, upper or lower limbs using standard trauma techniques. A plurality of straps and right and left plates form the posterior and lateral aspects of the splint.

In one embodiment, one end of each of the straps are passed superior to (over) the patient's fracture site and attached to the opposing end of each respective strap. A system of cushions and air bladders are mounted on the internal side of each plate. After the splint is in place on a patient, the air bladders are inflated to compress/stabilize the patient's pelvis. The straps and air bladders may then be further manipulated to optimize the fit and function of the splint.

The plates are designed to be of sufficient posterior-anterior height and rigidity such that the anterior straps project over, and are not in contact with the soft anterior abdominal/pelvic wall. This feature, and the inherent openness of a strap system relative to a solid piece of fabric or other material ensure that the providers can see and access the anterior abdomen, pelvis, and ano-genital regions without disengaging the device.

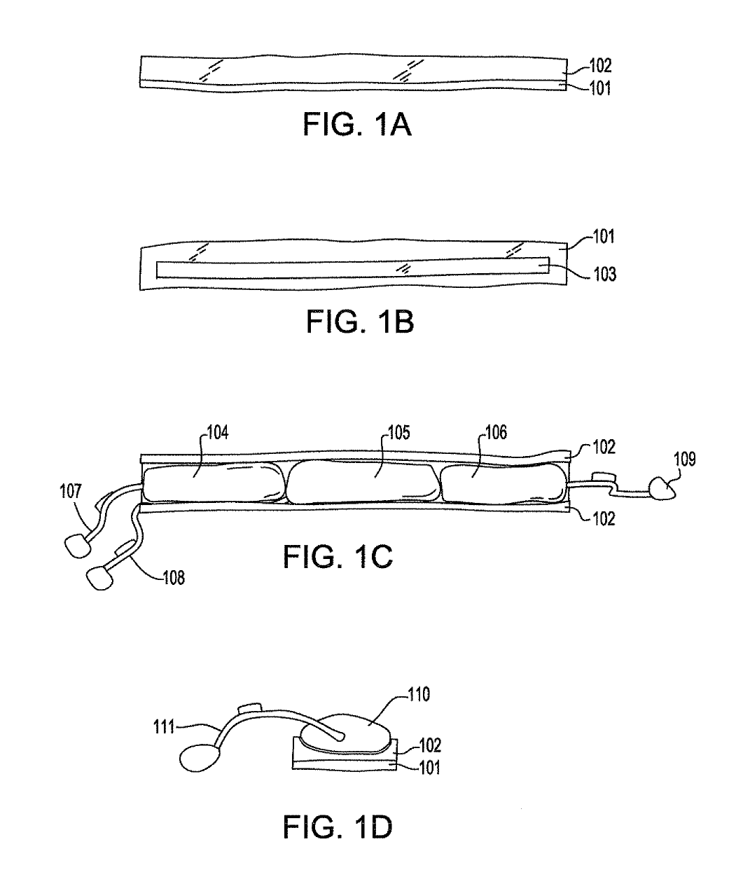

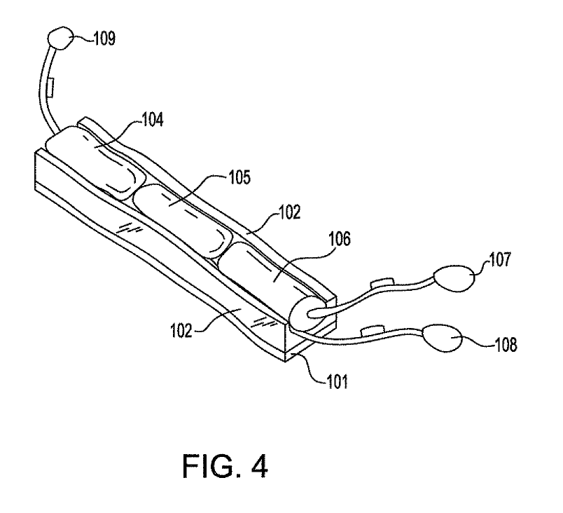

With reference to FIGS. 1 and 4, an embodiment of the plate assembly 100 will be described in detail. A plate assembly 100 includes a rigid plate 101 forming the base of the plate assembly 100. The rigid plate 101 is disposed with a Velcro™, i.e. hook and loop fastener, strip 103 on a bottom surface. A rigid foam padding 102 is bonded to a top surface of the rigid plate 101. The rigid foam padding 102 has a concave or ‘V’ shaped cavity on a top surface running lengthwise and dimensioned for accommodating one or more inflatable air bladders 104, 105 and 106. Additionally, Velcro™ straps can be added or remove as necessary to accommodate the particular needs of the physician and patient—such as for visualization and access to an area. It should be noted that use of rigid foam padding can be considered optional, as the inflatable bladders can be formed in a concave shape.

Each air bladder 104, 105 and 106 is respectively equipped with an air hose assembly, 107, 108 and 109 having an air hose, valve, pump and gauge. The air hose assembly 107, 108 and 109 allows adjustable inflation of each of the air bladders 104, 105 and 106 independently as needed to provide desired compressive force on an area of contact of the fracture site. The air pump may be battery operated or hand operated as is commonly used in blood pressure cuffs. As the air bladder 104, 105 and 106 is inflated, the rigid foam padding 102 directs the expansion of the air bladders 104, 105 and 106 outward in one direction, perpendicular to the plane of the plate assembly 100.

Moreover, the air pump may be integrated with the air bladder, or may be removable, in which case an air valve is provided to prevent air leakage from the air bladder 104, 105 and 106 once the air pump is removed.

Ribs (not shown) may be formed on the bottom outside surface of the plate 101 to increase rigidity of the posterior-anterior axis of the plate 101 so that the plate 101 does not bow over the fracture site as the straps 112 are attached and the air bladders 104, 105 and 106 inflated. Bowing would alter the compression vector of the device, cause compression of the soft anterior wall of the abdomen and pelvis, and limit access and visualization of the area, when applied to a pelvic fracture for example. Similar issues may arise from bowing when the compression splint is applied to spinal and long bone fractures as well. The ribs also form a reinforcing skeleton that strengthens the splint at contact point of the straps 112 with the plate assembly 100.

A preferred shape for the rigid foam padding 102 for use in a pelvic compression splint is as follows. The base thickness of the rigid foam padding 102 (at the inner concavity of the C) is dependent on the foam density, however the present invention has been prototyped with foam having a base thickness of between ½ inch and 1 inch. At the posterior edge of the rigid foam padding 102 a wedge preferably extends 2 extra inches medially and tapers back to baseline thickness two inches (in anterior height) up the splint, forming a triangular shape. Another wedge tapers out at a point preferably 7 inches in anterior height for the posterior edge.

This wedge preferably extends medially 2 inches from the baseline thickness of each rigid foam padding 102. It will reach this medial distance at 9 inches in anterior height. At that point each foam cushion remains at a constant 3 inches in thickness as it proceeds to the anterior edge of the plate 101. The specific dimensions provided are provided as exemplary dimensions only, which may be appropriate for pelvic fractures for example, but may not apply to reduction and alignment of long bone fractures.

For long bone treatment, three trapezoidal shaped slats with dimensions of 5″ and 4″ at the narrow ends and 10″ length are more advantageous. These dimensions are appropriate for an adult upper leg fracture. Smaller dimensions can be used for children and lower leg fracture.

Alternatively, in some applications ⅛ to 1 inch, and preferably ¼ inch padding if it is of an appropriate type and density may be used. In fact, in certain cases foam padding may not be needed at all, especially in the case of long bone fractures.

The air bladders 104, 105 and 106 provide strong compression and close approximation of the splint to the individual patient's external pelvic, limb or neck contours. The simplest arrangement would be similar to the large blood pressure cuffs and would have a single inflatable cushion housed in a nylon shell. The air bladders 104, 105 and 106 are fastened directly to the foam-lined internal surface of each splint using Velcro™ so that it is possible to remove the air bladders for maintenance and cleaning. The air bladders extend from the postero-medial to antero-medial ends of the “C” of the rigid foam padding 102. One example of rigid foam padding that is appropriate for use in the present invention is commonly known as memory foam

Using the inflatable compartment mounted in this fashion, the dominant compression vector is lateral to medial (medially-directed). The sides of the rigid foam padding 102 provide some anterior and posterior support.

When used for the treatment of pelvic fractures, the shallow C-shape of the rigid foam padding 102 and air bladders 104, 105 and 106 provide form-fitting “customized” compression along the entire span of the bony pelvis. Consequently, both anterior and posterior fractures (e.g. “open book” PS fractures and SI disruptions) are compressed and stabilized.

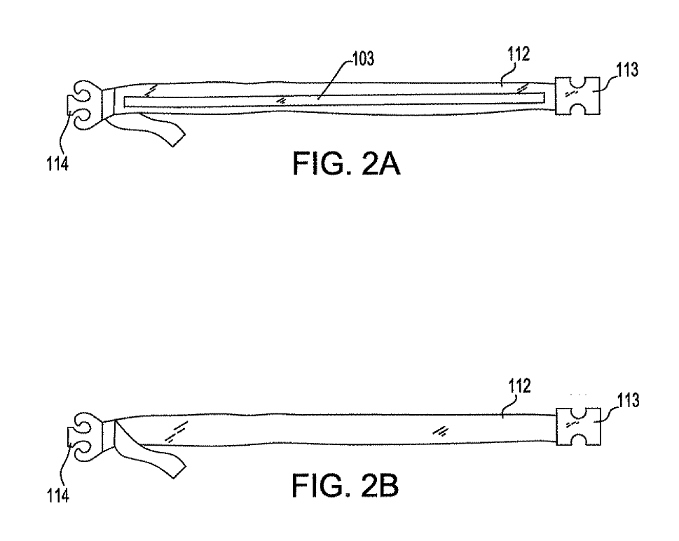

Referring to FIG. 2, a strap 112 for securing the above described plate assembly around a patient's long bone fracture in accordance with the present invention will be described herein. The strap 112 has a Velcro™ strip 103 running lengthwise on a first side of the strap 112. The Velcro™ strip 103 disposed on the strap 112 is of a type opposite to the Velcro™ strip 103 disposed on the plate assembly 100, such that contact between the two Velcro™ strips results in holding engagement between the strap 112 and the plate assembly 100. Additionally, each end of the strap 112 is provided with a female buckle end 113 and a male buckle end 114 adapted and configured for engaging with each other to form the strap 112 into a closed loop. Cordura® is an acceptable material for construction of the straps.

The strap 112 is adjustable in length so that each strap has a wide range of circumferential dimensions. In a preferred implementation, one strap 112 is affixed at a lower portion of the plate assembly 100, and a second at the upper portion. Additional straps 112 may be used as well if needed. It is important to note that the portions of the strap 112 which contacts the patient's skin have no seams or folds so that skin breakdown is minimized.

On most adults the strap 112 and plate assembly 100 extend inferiorly to superiorly from the buttock crease below the ischium to iliac crest which is located approximately in alignment with the third or fourth lumbar vertebral body, when applied to the pelvic region. For other bone fractures, placement of the straps is dependent on the specific location of and type of fracture being treated.

The plates perform a key function of the semi-rigid compression splint by conferring anterior-posterior rigidity. This rigidity allows the semi-rigid compression splint to be engaged without greatly compromising visual and operative access to the fracture site.

For applications of pelvic fractures, preferably, the plates measure 12″ inferior to superior by 11″ posterior to anterior (i.e. 11″ in anterior height). The 11″ anterior height is sufficient to clear the anterior abdominal wall of small, medium and large non-obese persons. For obese patients, it is possible to make plates with greater anterior height to accommodate an especially protuberant abdomen. Because the anterior height of the bony pelvis does not change as much as abdominal girth in the obese, the rest of the plate would not likely need significant alteration; the plates used for other fracture types are dimensioned as appropriate to properly accommodate the individual patient's physiology.

The plates 101 are preferably constructed of medical-grade plastic, such as that used in other pre-fabricated splints. Acrylic is preferred because it is inexpensive, strong, easy to work with, and readily available. Alternatively, it is possible that this rigidity could be provided using different materials, such as other types of plastic, or especially strong form-retaining inflatable cushions or inflatable/foam hybrids. Moreover, the components of the compression splint can be sterilized for use in operating rooms as well.

The plates 101 can employ ¼″ thick acrylic reinforced in the posterior-anterior plane by three ⅛″×1.25″×11″ ribs. These materials provide adequate splinting support and compression of the pelvis. The firm foam inner shell cushions 102, inflatable air bladders 104, 105 and 106 and adjustable strap 112 system combine to allow the compression splint to conform to the individual patient's pelvic contours.

However, the above material dimensions are provided as examples only. In practice many different sizes will be used in order to accommodate different patient age groups, sizes and body types. Moreover, the size and shape of the materials used are dependent on the particular fracture to which the compression splint will be applied.

In an embodiment of the present invention, the semi-rigid compression splint is provided with a complete set of plates, including different dimensions and shapes so that the compression splint can be customized in-situ for the specific patient physiology and fracture type.

Ideally, the straps are disposed and spaced to provide the strongest medially-directed compression forces at the sacro-iliac and pubic symphysis joints, in the case where the compression splint is applied to the pelvis. Different strap spacing is used for application of the splint to other fracture sites. Disruption of the sacro-iliac (SI) and pubic symphysis (PS) joints is the most common cause of life-threatening pelvic fractures. The straps should be positioned so as to avoid interfering with access to the anterior abdomen and pelvis and provide unnecessary and potentially undesirable compression of the iliac wings superior to the SI joint. The strap placement should also avoid external leveraging of the hip by the femur and to avoid unnecessary interference with access to the ano-genital area.

As discussed above, obese persons may require a splint with greater anterior height (e.g. 12″). This may be expected to change the dominant compression vector by lengthening the torque arm for the pull by the anterior trans-abdominal straps. Fortunately this effect can be attenuated by increasing the pulling force of the posterior sling by increasing the tension on the straps connecting it to the lateral plate.

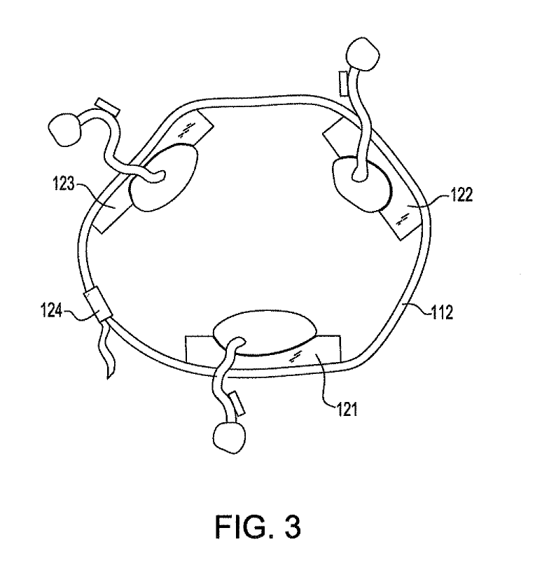

FIG. 3 illustrates an assembled compression splint in accordance with an embodiment of the present invention. As shown, three plate assemblies 121, 122 and 123 are disposed along a looped strap 112 fastened with a buckle 124. In the particular arrangement shown, a first plate assembly 121 is positioned on the posterior side of the fracture, while a second and third plate assembly 122 and 123 can be positioned at either end of the anterior portion of the fracture.

As shown in FIG. 3, the compression splint of the present invention applies compressive and rotational force three-dimensionally. This allows for a more controlled reduction of a fracture, since the compressive and rotational forces applied to the fracture by each inflatable air bladder can be varied independently, as necessary.

Proper fracture reduction is necessary for fracture reduction and stabilization, control of bleeding, and promotion of proper healing of the bone, as well as preventing bone deformities and reducing pain. The present invention is appropriate for use in indirect reduction of fractures. When reducing or setting a fracture of a long bone, the compression splint is applied to the fracture site and compressive and rotational force is applied as necessary, by varying the inflation of the individual bladders, to realign the bones into the proper position. Since the reduction is performed using the compression splint, no additional splint or cast is required. Consequently, the chance of the reduced bones shifting out of alignment while a cast is being formed around the injury site is eliminated.

Moreover, once the fracture has been reduced with the compression splint X-rays can be taken of the site to verify that the bones are properly aligned. If the X-rays indicate that the bones are not properly aligned or not aligned to the optimal position, the compression splint can be readjusted without much effort.

In contrast conventional casts would need to be broken and reapplied in order to adjust the bones. In the conventional fracture treatment using external compression frames, some imaging technology, for example magnetic resonance imaging, cannot be used due to the high metal content of both the compression frames and securing pins used. The compression splint of the present invention, when used for fracture reduction can be worn by the patient for the duration of the bone-mending process.

Additionally, the gaps between the inflatable air bladders of the compression splint allow for insertion of orthopedic hardware into the bones once the initial fracture reduction is performed with the compression splint. Consequently, the compression splint can also be used in open reduction procedures.

The present invention, however, allows for any number of plate assemblies to be employed to further customize the compressive force applied to a patient's pelvis or long bones; and for the positioning of the individual plate assemblies to be adjusted as necessary. Moreover, plate assemblies of various widths may be used as well. The utilization of Velcro™ on the interior surface of the strap allows easy repositioning, removal or addition of plate assemblies.

Alternative inflatable air bladder designs can also be utilized. It is possible to make several more complex variations of the inflatable air bladder. For example, the air bladders may be molded into a sharper C-shape, or be ribbed so that it conforms more closely to the pelvis or other fracture site. Alternatively, two or more compartments may be housed in each nylon shell so that compression in each compartment may be changed.

If longer term use of the splint is necessary, a system of alternatively inflating compression compartments (e.g. such as is currently employed in Venodyne deep venous prophylaxis systems) could be used so that skin breakdown is minimized. The timing of inflation and release of these compartments would have to be adjusted so that even compression is maintained at the fracture.

Lower level trauma centers and EMS companies may find it a desirable means for performing transfer of a pelvic fracture patient to a distant level one trauma center after initial assessment and stabilization is performed. Again, the versatility, low-cost, simplicity, and speed of deployment make the semi-rigid compression splint of the present invention likely to become a very popular, effective and widely used product with potential to become standard equipment for EMS systems, emergency departments and trauma units throughout the U.S. and other countries.

Although external frame systems have great benefits, they are not widely used because of their disadvantages, which mainly consist of limiting the examination of the patient, delaying other effective treatment modalities, causing device-specific skin, lung and infectious complications, and requiring extra time or expertise in early trauma management. Because the semi-rigid compression splint of the present invention has the potential to provide the same benefits as these devices with less medical, temporal and economic costs, its widespread use is likely, including in military settings, rural and community health systems, athletic competitions, and even developing nations, in much the same way that cervical collars have become prevalent.

Descriptions of dimensions and directions herein employ standard anatomical terminology, including lateral, medial, anterior, posterior, inferior, and superior. These terms relate to a given object's position in reference to the supine patient's body (i.e. objects relatively closer to the head are superior to objects closer to the feet, and objects relatively closer to the abdominal surface are anterior to those that are closer to the patient's back). Common terms such as “above”, “below”, “over”, etc. are also used for convenience in a conventional non-anatomic sense and refer to the position of objects in space.

Size specifications for the semi-rigid compression splint of the present invention are intended to provide guidelines for a “medium-sized” final product that will fit most adults. They were obtained by measuring the pelvic dimensions of several medium-sized males and by reviewing male and female cross-sectional pelvic anatomy in a text-book with a scale ruler.

Specifications of the composition of the semi-rigid compression splint of the present invention are based on a familiarity with other emergency medical products, local availability of materials, and the facility for working with these materials. It should be apparent to those skilled in the art that different materials may be chosen to construct the semi-rigid compression splint without departing from the scope or spirit of the present invention.

Moreover, the compression splint of the present invention can be utilized to exert compressive and rotational force on long bones and extremities, as well. When used on a long bone or extremity, such as an arm or leg, the compression splint can be situated to exert compressive and rotational forces on the long bones, and stabilize the fractured or injured bones.

Additionally, the compression splint can also provide compressive force to arteries and veins located in extremities in order to staunch bleeding in a manner similar to a conventional tourniquet. If provided in larger dimensions, the compression splint of the present invention can further be used on a thorax portion of the body, for example to stabilize fractured ribs, etc.

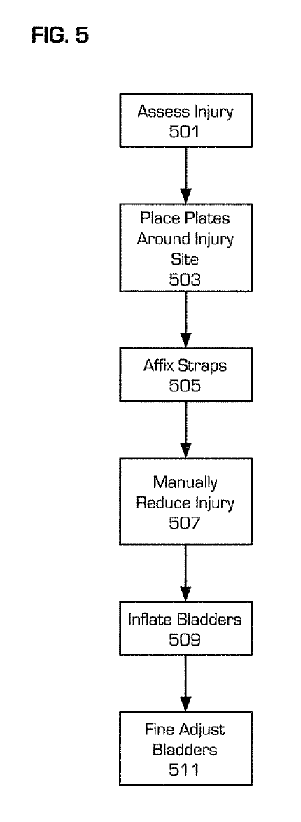

Referring to FIG. 5, a flow diagram is illustrated representing the steps for utilizing an embodiment of the present invention for stabilizing and reducing a fracture of an extremity, such as a fracture of the arm or leg. Initially, the medical technician identifies the location and type of fracture in step 501. Once the injury has been assessed, the medical technician proceeds with reduction of the fracture by placing the three plates around the fracture area in step 503. In step 505, the medical technician affixes straps to the plates to hold the plates in place. The medical technician proceeds with manually reducing the fracture, and or dislocation, in step 507.

Once the injury has been manually reduced, the bladders situated on the plates are inflated so as to hold the reduction in step 509. Additionally, at this point the reduction can be fine tuned by adjusting the inflation levels of each bladder in step 511. The compression splint can be left in place in this state until a definitive fixation of the injury is performed.

The described embodiments of the present invention are intended to be illustrative rather than restrictive, and are not intended to represent every embodiment of the present invention. Various modifications and variations can be made without departing from the spirit or scope of the invention as set forth in the following claims both literally and in equivalents recognized in law.

What is claimed is:

1. A compression splint for reduction, compression and stabilization of fractures, said compression splint comprising:
a plurality of rigid plates selectively disposed at a plurality of position of a fracture site;
a plurality of inflatable cushions, each cushion of said plurality of cushions positioned between respective plates of said plurality of plates and said fracture site; and
a support means for supporting the plurality of plates and plurality of cushions compressively against said fracture site in three dimensions.

2. The compression splint as in claim 1, wherein each inflatable cushion of the plurality of inflatable cushions has individually adjustable inflations.

3. The compression splint as in claim 1, wherein at least a subset of the plurality of rigid plates is removable.

4. The compression splint as in claim 3, wherein the plurality of rigid plates is provided in a variety of dimensions for accommodating a range of fractures.

5. The compression splint as in claim 1, wherein the support means comprises at least one strap including an engaging assembly for holding opposite ends of the strap together.

6. The compression splint as in claim 1, wherein the compression splint is dimensioned and adapted for use on long bone fractures.

7. The compression splint as in claim 1, wherein the compression splint is dimensioned and adapted for use on fractures of a pelvis.

8. The compression splint as in claim 7, wherein the support means is disposed to allow visualization and access to anterior abdomen, pelvis and ano-genital regions of the pelvis.

9. The compression splint as in claim 7, wherein the plurality of rigid plates are dimensioned to provide sufficient height and rigidity to prevent contact between the support means and a soft anterior abdominal/pelvic wall.

10. A compression splint for reduction, stabilization and compression of a long bone or an extremity, said compression splint comprising:
a plurality of rigid plates selectively disposed at a plurality of position of said long bone or extremity, a number of said plurality of plates being removable;
a plurality of inflatable cushions, each cushion of said plurality of cushions positioned between respective plates of said plurality of plates and said long bone or extremity; and
a support means for supporting the plurality of plates and plurality of cushions compressively against said long bone or extremity in three dimensions.

11. The compression splint as in claim 10, wherein each inflatable cushion of the plurality of inflatable cushions has individually adjustable inflations.

12. The compression splint as in claim 10, wherein the plurality of rigid plates is provided in a variety of dimensions for accommodating a range of extremity dimensions.

13. The compression splint as in claim 10, wherein the support means comprises at least one strap including an engaging assembly for holding opposite ends of the strap together.

14. A pelvic compression splint for splinting support and compression of a pelvis, the pelvic compression splint comprising:
a plurality of rigid plates selectively disposed at a plurality of position of the pelvis, a number of the plurality of plates being removable;
a plurality of inflatable cushions, each cushion of the plurality of cushions positioned between respective plates of the plurality of plates and the pelvis; and
a support means for supporting the plurality of plates and plurality of cushions compressively against the pelvis in three dimensions.

15. The pelvic compression splint as in claim 14, wherein each inflatable cushion of the plurality of inflatable cushions has individually adjustable inflations.

16. The pelvic compression splint as in claim 14, wherein the plurality of rigid plates is provided in a variety of dimensions for accommodating a range of pelvic dimensions.

17. The pelvic compression splint as in claim 14, wherein the support means comprises at least one strap including an engaging assembly for holding opposite ends of the strap together.

18. The pelvic compression splint as in claim 14, wherein the support means is disposed to allow visualization and access to anterior abdomen, pelvis and ano-genital regions of the pelvis.

19. The pelvic compression splint as in claim 14, wherein the plurality of rigid plates are dimensioned to provide sufficient height and rigidity to prevent contact between the support means and a soft anterior abdominal/pelvic wall.
